(19) United States
(12) Patent Application Publication
(10) Pub. No.: US 2010/0036284 A1
(43) Pub. Date: Feb. 11, 2010

(54) Guide-Wire and Guiding Insert Placement Assembly for Over-the-Wire Catheter Placement and Method of Use

(75) Inventors: Derek Charlton Laynes, Parkside (AU); Ivan Anthony Curtis, Vale Park (AU); Donald Philip Chorley, Parkside (AU)

Correspondence Address: THE WEBB LAW FIRM, P.C., 700 KOPPERS BUILDING, 436 SEVENTH AVENUE, PITTSBURGH, PA, 15219, US

(73) Assignee: MICRONIX PTY LTD, Parkside (AU)

(21) Appl. No.: 12/440,372

(22) PCT Filed: Sep. 7, 2007
(86) PCT No.: PCT/AU07/01332

§ 371 Date: Mar. 6, 2009

(30) Foreign Application Priority Data
Sep. 8, 2006 (AU) 2006904933

(51) Int. Cl. A61M 25/09

(52) U.S. Cl. 600/585

(57) ABSTRACT

A guiding insert assembly is disclosed that has electromagnetic-radiation-elements at each of its ends. A guiding insert having these elements is suitable for use in conjunction with known equipment in the process of locating the distal end of the guiding insert being placed into a patient. The proximally located element of the guiding insert can be used instead of a connector for contact-less coupling of the guiding insert and the location determining equipment, using an inductive coupler, so as to couple signal to and from the guiding insert during the location process. The coupling device preferably includes an insert retention arrangement, a guiding insert identification means and an end of travel detector. The placement of a catheter over the guiding insert either after placement or at the time of placement is possible.

This invention relates to placement of catheters using a location device and includes the coupling between a locatable guiding wire or guiding insert and the location device.

### INCORPORATION BY REFERENCE

Australian Provisional Patent application number 2006904933 filed 8 Sep. 2006 entitled GUIDE-WIRE AND GUIDING INSERT PLACEMENT ASSEMBLY FOR OVER-THE-WIRE CATHETER PLACEMENT AND METHOD OF USE in the name of Micronix Pty Ltd is hereby incorporated by reference to this specification.

### BACKGROUND

Placement of catheters into the body for therapeutic, diagnostic and interventional purposes including the delivery and capture of fluids is an important part of the treatment and rehabilitation of patients afflicted with many types of medical conditions.

Numerous drugs are also carried in catheters by gases and liquids.

Liquid nutrients for patients can also carried by catheters for enteral and parental feeding.

Fluids from inside the body of a patient can also be captured by a catheter including blood and other bodily fluids as well as gasses from the digestive tract and lungs.

Pressure, temperature and pH measurements are often recorded via inserted catheters. Interventional procedures such as balloon dilatation and cardiac ablation procedures are also performed via catheters. Accurate placement of the catheters to perform these functions is essential in order for these procedures to succeed.

An example of an interventional procedure is angioplasty in which a physician threads a balloon-tipped catheter over a guide wire to the site of a narrow or blocked artery and then inflates the balloon to open it. The balloon is then deflated and removed from the artery. Vascular stent placement, which is often performed at the same time as an angioplasty, involves the placement of a small wire mesh tube called a stent; the stent is guided over the wire and within the lumen of a catheter to the area of concern in the newly opened artery. The stent is a permanent device that is left in the vasculature.

Another example of a therapeutic procedure is embolization, which is a method of occluding one or more blood vessels in instances in which their continuing patency may be harmful to a patient. An occluding material is passed through a catheter, which has been guided to the targeted vessel by being threaded over a guide wire so that its distal tip is positioned within the vessel to be occluded. Therapeutic embolization may also serve to eliminate an arteriovenous malformation (AVM), an abnormal connection between an artery and a vein.

The type of catheter used may be named according to the bodily system within which it is used and its site of insertion, or for the function it provides for example, the delivery or extraction of fluids to and from the body. For example, an intravascular catheter is a device that consists of a tube slender enough to be inserted in the patient's vascular system for short-term use (less than 30 days). One example is a central venous catheter (CVC), which is a flexible tubular device, placed within a vein whose distal end is intended to be located within the vena cava (inferior or superior). A Peripherally Inserted Cardiac Catheter (PICC) is one that is inserted in a peripheral vein, e.g. in the arm, and then advanced until the distal end is located into the superior vena cava.

Catheters are also used for the delivery of drugs to tumours, through the vasculature to tissues and organs.

The correct placement of the distal end (the leading tip of the catheter inserted into a patient) into the appropriate terminal position within a patient can be critical to successful treatment of the patient.

Placing various catheters to their desired location can involve the use of a wire that is separately inserted along the path intended for the catheter in advance of its insertion. Use of a guiding wire thereby expedites insertion of catheters into sites of access that pose clinical difficulties and access along paths of placement and to target sites that are difficult to access, or are otherwise inaccessible using a catheter alone. Guide wires are essential to the use of certain catheters. They may be configured in the form of single or multiple strands of wire. When configured in multiple strands, the guide wire may be made of more than a single type of material. Guide wires are made of materials that are known to be biocompatible with patients even though they are only generally inside the patient temporarily.

Stylets are generally used to stiffen catheters during placement procedures by being located in the catheter before the catheter is placed into the patient.

In contrast to the general use of a stylet guide wires are inserted prior to catheter placement so that the catheter can be guided to the intended site within the body when placed over the guide wire.

Thus, despite these typical use differences the terms stylet and guide wire are sometimes used interchangeably.

Furthermore, a guiding insert placement assembly also refers to a guiding insert which can use stylets and guide wires which incorporate a receiving or transmitting element such as an electromagnetic-radiation-element that is used in conjunction with a location device, to detect or indicate the general position or a relative position of the receiving or transmitting element in the patient and thus assist the clinician to property locate the catheter for its purpose.

Location devices that utilize electromagnetic technologies typically provide a system to locate the distal end of a catheter and in particular there are those that can be used to display that location and relative movements of the distal end of the catheter at the patient's bedside. These devices are used as an adjunct or as an alternative to methodologies that create images of the actual patients and the locations of catheters, particularly their distal ends which are not readily available at the patient's bedside.

Although imaging equipment is commonly used to confirm that catheters have been placed correctly, determination of the spatial location of a catheter or its distal end within the patient are not always accurate since imaging must be obtained from at least two perspectives. In particular, technologies that provide both a frontal and a lateral or side view of the catheter or just its distal end are desirable but this often requires transportation of the patient to the imaging equipment and sometimes movement of the patient while being imaged. Patient movement can understandably be the source of discomfort and inconvenience to both the patient and the medical staff.

Technology described in U.S. Pat. No. 5,099,845 ('845) titled Medical Instrument Locating Means in the name of the applicant is one such means by which devices are useable at the bedside for locating the distal end of a catheter in a patient and the disclosure of that specification is incorporated by reference into this specification.

A further example is U.S. Pat. No. 4,905,698 ('698) titled “Method and Apparatus for Catheter Location Determination” to Strohl et al.

In both above examples of bedside catheter location arrangements, the catheter is fitted with an electromagnetic-radiation-element on its distal end and in the '845 patent the element transmits electromagnetic energy and in the '698 patent the element receives electromagnetic energy.

PCT/AU2006/000027 (WO2006/074510) titled “Guiding Insert Assembly for a Catheter used with a Catheter Position Guidance System” is also in the name of the present applicant and the disclosure of that specification is incorporated by reference into this specification. It will be noted that a guiding insert, a generic term used in that specification, may include within its scope a guide wire or a stylet, which is fitted inside a catheter as the catheter is being inserted into the body of the patient. In such an embodiment, the guiding insert is used to stiffen the catheter which would otherwise be too pliable to make possible the task of directing the distal end of the catheter to its desired location within the body. A guiding insert that includes such a stiffening wire may also be referred to as a guiding stylet.

Since the electromagnetic-radiation-element can be fitted on to a guiding insert instead of, at much greater expense, into or onto the distal end of a catheter, the guiding insert serves dual functions, i.e. as a device to confer the appropriate degree of rigidity to a catheter and also as a device to which the electromagnetic-radiation-element is attached that is positioned at the distal end of a catheter. A function of the guiding insert of this type is as a carrier for an antenna for either signal transmission or reception, which when used with suitable catheter location technology, enables a user to navigate tortuous and restricted passageways within bodily systems using the stiffness of the guiding insert with the assistance of the catheter location technology.

In order to reach a desired location within a patient, such as the site of a tumour a guiding insert may be used to not only access the vasculature leading to an organ but also to traverse vessels within an organ. Ancillary use of a guiding inserts in this way enables catheters to be placed into desired locations within patients.

A technique widely known as the Seldinger technique involves the introduction in to a vein or artery of a very thin wire (guide wire) through the bore of a needle shortly after the initial puncture of the vein or artery. The Seldinger technique has been developed, according to the experience and choice of equipment of operators into a number of variants that are collectively referred to as the ‘Seldinger Technique’, or a modification thereon. Following satisfactory venipuncture, the needle is then removed, leaving the guide wire within the vein or artery so that it can be pushed gently through the cardiovascular system via the most expedient path to the desired site within a patient's body. The guide wire can be manipulated by twisting, pulling and pushing into location. Once the user is satisfied that the guide wire is in the optimal position, the location of the guide wire's distal end and the path traversed to reach the location can be verified by the types of imaging methodologies referred to earlier.

When the clinician has confirmed that the distal end of the guide wire has been advanced into the desired location, a catheter is slid over the guide wire from its proximal end and is passed over the wire until the catheter's distal end has been advanced sufficiently that it reaches as far as but no further than the distal end of the guide wire.

Confirmation of correct placement by means of radiological imaging requires scheduling of resources and transportation of the patient or equipment. A guide wire may also assist clinicians because of its radiopacity. For this reason guide wires are typically left within catheters temporarily while radiographic images are taken.

Images must be reported on by highly qualified specialists prior to use of catheters for their intended purposes.

Procedures that require radiology to guide placement of catheters to areas targeted for interventional and diagnostic procedures often require intensive use of radiation. These techniques, which may entail multiple doses of x-rays, increase considerably the exposure to radiation of both patients and the attending medical staff.

The location devices that are described above, which are used with patients either in place of or as an adjunct to image-taking can reduce considerably the radiation dose persons involved in catheter placement procedures. These catheter location devices can only be used if either the catheters or the guiding inserts (guide wires or stylets) that are used are fitted with a suitable electromagnetic-radiation-element. Sensors that do not contain a electromagnetic-radiation-element may be referred to as transducers. Transducers can be used to detect one or more characteristics such as temperature, pressure, etc. of the environment into which they are placed.

Transducers may be fitted to catheters at their tips, within the lumens of catheters, or to stylets, guide wires or other devices within catheters. Such transducers may be intended to be removable or not.

In not only the technologies described that employ location of catheters or guiding inserts fitted with an electromagnetic-radiation-element, but also in technologies that do not employ electromagnetic-radiation-elements, transducers may require an electrical connection between the distal and proximal ends of the devices attached to or fitted inside catheters placed into the body, as do catheter location technologies.

In the same way that for catheter location devices the proximal end would require an electrical connector that allows the element and/or a transducer to be connected to external equipment including a signal generator, or for some location devices, to be connected to a signal detector, both of which may enable determination of the location of the distal end of the catheter or guiding insert, electrical coupling of a transducer intended to detect or measure a physiological parameter may be required between the proximal end of the device and external equipment.

However, the outer diameter of an electrical connector will not be able to be made small enough to fit inside the lumen of a catheter, especially those catheters used in paediatric patients and hence use of a catheter with an over-the-guide-wire placement technique may not be possible.

For the electrical connector to pass through the lumen of a catheter, the internal diameter of the catheter lumen must be larger than the maximum outer diameter of the connector. If that is not the case the guiding insert needs to be at least twice as long as the catheter for it to be sufficiently long that the full length of the catheter can be accommodated over that portion of the guiding insert, free of the length of the inserted guide wire that would be inserted into a patient's body. The problem of connector size referred to above would add significant cost, whereas the additional length of guiding inserts imposed by over-the-wire placement techniques would complicate and pose possibly insurmountable issues regarding maintenance of asepsis during procedures.

In any case, for both technologies other than catheter location and catheter location technologies, regardless of the electromagnetic-radiation-element's configuration as either a transmitter or receiver, for signal processing to be possible, the electrical connector must couple with a complementary connector in the catheter location system in a reliable, safe, aseptic and convenient manner. The achievement of these requirements for a guiding insert is difficult and expensive relative to the cost of a catheter. Potentially a connector may be comparatively expensive relative to other components of the guiding insert and catheter apparatus and thereby impose a substantial cost burden on the complete catheter assembly. The same factors of convenience and cost may also apply for technologies other than catheter location devices that seek to sense one or more characteristics at the distal end or along the length of a catheter inserted into a patient.

Electrical connectors attached to the proximal end of guiding inserts pose problems and the various embodiments of the invention disclosed herein provide an alternative or at least provide a choice when using a guiding insert in a catheter to which an electrical connection must be made to support the function of a device located at the distal end or along the length of the guiding insert.

### BRIEF DESCRIPTION OF THE INVENTION

A guiding insert assembly having a distal end and a proximal end including
a distally located electromagnetic-radiation-element;
a proximally located electromagnetic-radiation-element; and
a conductive wire arrangement connecting the distally located electromagnetic-radiation-element to the proximally located electromagnetic-radiation-element.

A guiding insert signal inductive coupling arrangement including
a guiding insert assembly having a distal end and a proximal end including
   a distally located electromagnetic-radiation-element;
   a proximally located electromagnetic-radiation-element; and
a conductive wire arrangement connecting the distally located electromagnetic-radiation-element to the proximally located electromagnetic-radiation-element; and
an inductive coupler associated with a signal receiver or generator apparatus adapted to receive the proximal end of the guiding wire to permit inductive exchange of electromagnetic energy between the coupler and the proximally located radiation element for transmission to the distally located electromagnetic-radiation-element.

In a further aspect of the invention the maximum outer diameter of both electromagnetic-radiation-elements is substantially the same or different.

In yet a further aspect of the invention the maximum outer diameter of the conductive wire arrangement is less than the maximum outer diameter of both electromagnetic-radiation-elements.

In an aspect of the invention the maximum outer diameter of either of the electromagnetic-radiation-elements is sized so as to allow a catheter to pass over at least the proximal electromagnetic-radiation-element or both of the electromagnetic-radiation-elements.

In another aspect of the invention the distal end of the guiding insert assembly is atraumatic in its effect upon the tissue of a patient in whom the guiding wire assembly is placed.

In yet a further aspect of the invention the guiding insert assembly further includes at either or both the proximal end and distal end an electromagnetically powered identification device.

In yet a further aspect of the invention the guiding insert assembly further includes at the proximal end an optical identification device.

In yet a further aspect of the invention the optical identification device at the proximal end of the guiding insert assembly further includes a self-test means to verify that the identification device is working correctly.

In another aspect of the invention the guiding insert assembly further includes indicia along at least a portion of the length of the conductive wire arrangement to indicate the distance from the distal end as gauge of the location of the distally located electromagnetic-radiation-element.

In an aspect of the invention the guiding insert assembly further includes indicia on the proximal end that identifies a type of guiding wire assembly.

In a further aspect of the invention the indicia includes a colour, shape, relative position of colour and/or shape or a combination of those indicia.

In a further aspect of the invention the guiding insert signal inductive coupling arrangement further includes a position detector for the proximal end of the guiding wire assembly.

In a yet further aspect of the invention the position detector provides a human detectable signal of the positioning of the proximally located electromagnetic-radiation-element so as to permit inductive exchange of electromagnetic energy between the coupler and the proximally located electromagnetic-radiation-element.

In another aspect of the invention the guiding insert signal inductive coupling arrangement further includes a measuring device that detects a voltage or current of the inductive coupler device to provide an indication of predetermined characteristics of the guiding wire assembly.

In another aspect of the invention predetermined characteristics include:
guiding wire material type;
resistance of conductive wire assembly; or
location of the proximally located electromagnetic-radiation-element with respect to the inductive coupler device.

In another aspect of the invention the guiding insert signal inductive coupling arrangement further includes an impedance measuring device that detects an abnormality in the guiding wire assembly, including an open circuit or short circuit of the inductive coupler or with the electromagnetic-radiation-element in the guiding wire assembly.

In another aspect of the invention the guiding insert signal inductive coupling arrangement further includes an impedance measuring device that detects a characteristic of the electromagnetic-radiation-element of the guiding wire assembly, indicative of the efficacy of coupling of the inductive coupler with the inductive coupling.

In another aspect of the invention the guiding insert signal inductive coupling arrangement further includes an impedance measuring device that detects an impedance related abnormality in the material of the guiding wire assembly.

In another aspect of the invention the guiding insert signal inductive coupling arrangement wherein if an abnormality is detected by the impedance measuring device, the inductive coupling arrangement ceases inductive exchange of electromagnetic energy between the coupler and the radiation element.

The reference to any prior apparatus or method in this specification is not, and should not be taken as acknowledgment or any form of suggestion that such prior apparatus or method forms part of the common general knowledge.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

A detailed description of one or more preferred embodiments of the invention is provided below along with accompanying figures that illustrate by way of example the principles of the invention. While the invention is described in connection with such embodiments, it should be understood that the invention is not limited to any single embodiment. On the contrary, the scope of the invention is limited only by the appended claims and the invention encompasses numerous alternatives, modifications and equivalents. For the purpose of example, numerous specific details are set forth in the following description in order to provide an understanding of the present invention.

### BRIEF DESCRIPTION OF THE FIGURES

FIG. 1 depicts a perspective view of an embodiment of a guide wire according to the invention having electromagnetic-radiation-elements at each end thereof;

FIG. 1A depicts an expanded perspective view of an electromagnetic-radiation-element of FIG. 1;

FIG. 1B depicts an expanded perspective view of the connecting guide wire of FIG. 1;

FIG. 2 depicts the guide wire of FIG. 1 in use during placement of its distal end in a patient and also having its proximal end located in an inductive coupler associated with a signal receiver or generator apparatus;

FIG. 3 depicts the guide wire of FIG. 1 in use having a catheter placed over its proximal end for placement into a patient;

FIG. 4 depicts an over the guide wire placement of a catheter while the distal end of the guide wire is at a desired location within the patient;

FIG. 5 depicts a catheter in place within a patient ready for use;

FIG. 6 depicts a functional illustration in cross-section of one embodiment of an arrangement to locate the proximal end of the guide wire in an inductive coupler which is part of a locator device used to indicate the location of the distal end of the guide wire in a patient;

FIG. 7 depicts a detailed illustration of an embodiment of the arrangement to locate the proximal end of the guide wire within an inductive coupler which is part of a locator device and an equivalent circuit;

FIG. 8 depicts a detailed illustration of the inductor bobbin;

FIG. 9 depicts an equivalent circuit to an inductive coupler arrangement;

FIG. 10 depicts a signal driver circuit for supplying a signal to the inductive coupler that provides a signal for electromagnetic radiation from the distal end of the guide wire;

FIG. 11 depicts a pulse width modulation timing scheme suitable for driving the electromagnetic-radiation-element; and

FIG. 12 depicts a pulse width modulation signal driving circuit for use with an embodiment of the invention.

### DETAILED DESCRIPTION OF EMBODIMENTS OF THE INVENTION

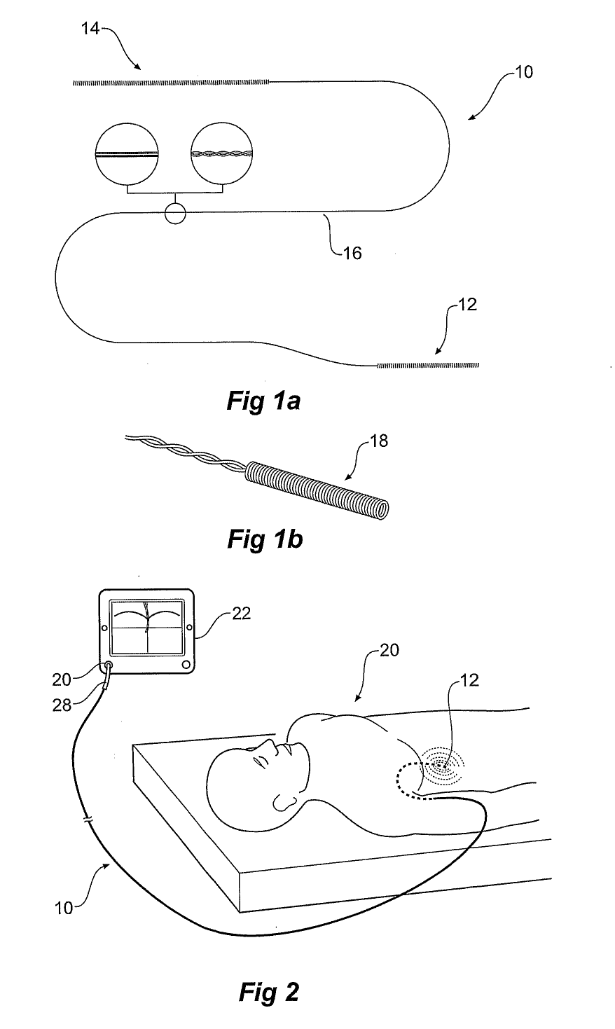

A guiding insert assembly 10 is depicted in FIG. 1 having a distal and a proximal end referred to with respect to the in use positioning wherein the distal end is inserted in a patient and the proximal end is insertable into a signal receiver or generator apparatus. The assembly includes a distally located electromagnetic-radiation-element 12; a proximally located electromagnetic-radiation-element 14; and a conductive wire arrangement 16 connecting the distally located electromagnetic-radiation-element 12 to the proximally located electromagnetic-radiation-element 14. Preferably the distal end of the guide wire is atraumatic in its effect upon tissue in order that damage does not occur to any of the vessels or organs that are traversed while the guiding insert assembly is being placed by the clinician.

Each electromagnetic-radiation-element of the guiding insert depicted in FIG. 1 is sized to have a maximum outer diameter that is substantially the same along its full length. An electromagnetic-radiation-element 12 or 14 is depicted in FIG. 1A as coiled wire 18, although the electromagnetic-radiation-element could be of any different configuration but still suitable to radiate or receive electromagnetic energy. It is also possible for the electromagnetic-radiation-element to be a device that is capable of receiving and radiating electromagnetic energy that is, for example, a passive micro-strip arrayed on a non-conductive substrate, or in a further example, a circuit configured on an integrated substrate having at least one radiating portion. There are a variety of configurations and devices that are suitable for performing the function of an electromagnetic radiating element.

It should also be noted that although the invention is illustrated in this document by having the electromagnetic element 12 of this embodiment as a radiating element, the electromagnetic element could be a receiving element. Of course the other electromagnetic element of the assembly would be required to correspondingly receive or transmit accordingly, so as to support the operation of the apparatus.

In any event the maximum outer diameter of the electromagnetic-radiation-element is preferably equal to or less than the maximum outer diameter of the conductive wire arrangement 16.

Further, the wire used to form the guiding insert assembly is preferably a single continuous wire because the electromagnetic energy received at one end will be conducted to the opposite end and radiated. Yet further, any break in the continuous wire will be capable of detection so that use of the guiding insert for location purposes can be monitored for safety and surety reasons.

Furthermore, it is preferable for the guiding insert to be made from materials that perform the same or similarly to guides wire currently used that do not have electromagnetic radiation capabilities. Such materials can include stainless steel, tempered steel, biocompatible materials and are configured so that the fully assembled device is biocompatible, should there be any risk of non-biocompatibility of any other component parts. Materials from which the guiding inserts are made are preferably also capable of undergoing multiple sterilisation cycles, (although any re-use of a guide wire is not necessarily recommended) and if so only in accordance with agreed and approved institutional and regulatory protocols.

The coils 12 and 14 depicted in FIG. 1 are conductively connected to each other with two electrically conductive wires depicted having twists suitable to provide self-support so that the guiding insert can be pushed, pulled and twisted at the proximal end to navigate it through the body.

The two wires provide a continuous electrically conductive circuit that permits transmission of electromagnetic signals from one end to the other of the guiding insert assembly 10. As will be described in greater detail later in the specification, the conductivity of the guiding insert can be monitored such that any discontinuity can be detected so that the user can be alerted and has the option to cease use of the location device in such an event.

FIG. 2 depicts the guide wire 10 of FIG. 1 in use during placement of its distal end 12 in a patient while its proximal end 14 (covered by a disposable sheath 28) is located in an inductive coupler assembly 20 associated with a signal receiver or generator apparatus 22.

The guide wire is directed by the clinician (not shown) into the patient 20 through appropriate passage ways until the distal end of the guide wire 12 is approximately in the desired location within the patient. As stated previously the access methodology is a clinical choice.

To assist placement of the distal end of the guide wires in patients, a guidance apparatus of the kind disclosed in U.S. Pat. No. 5,099,845 and the apparatus and method disclosed in PCT/AU2006/000027 (WO2006/074510) both in the name of the present applicant can be used. Although not exclusively so, as any apparatus or method that uses electromagnetic radiation transmission or reception to or from an electromagnetic-radiation-element located at the distal end of the guide wire will suffice. The guide wire 10 of this embodiment is equipped with such an arrangement in the form of a coil but as discussed elsewhere the actual radiation element is variable in form.

The inductive coupler assembly 20 associated with a signal receiver or generator apparatus 22 will be described in greater detail later in this specification. Suffice to state at this part of the specification that there is a contact-less connection between the proximal end of the guide wire and the signal receiver or generator apparatus.

The remainder of any apparatus associated with the location arrangement is not depicted in the figures but may include a display for assisting the clinician guide the distal ends of the guiding insert to desired locations in patients.

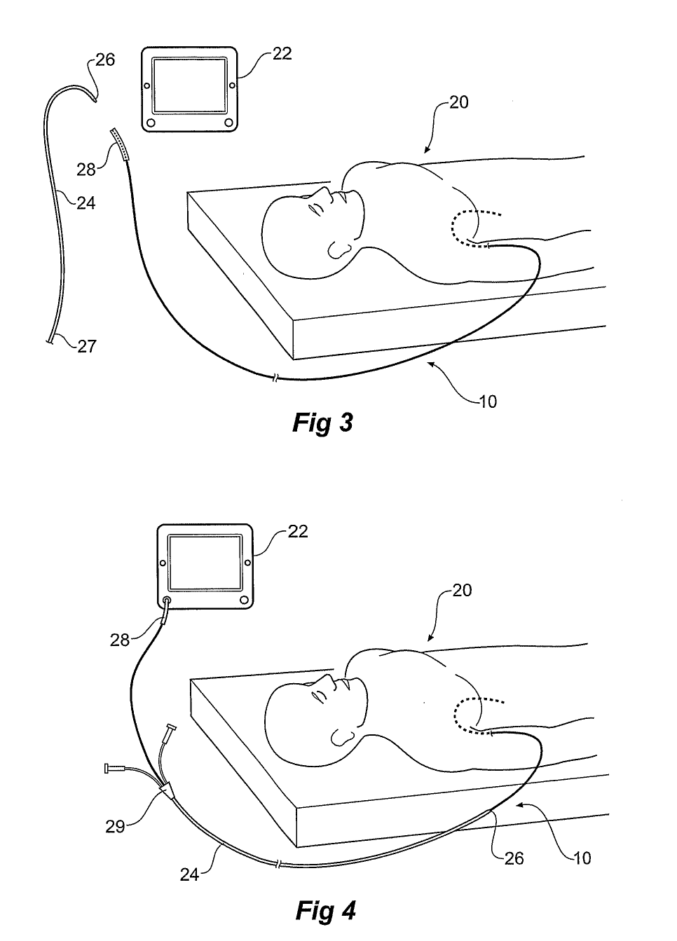

FIG. 3 depicts the guiding insert of FIG. 1 in use having a catheter 24 placed over its proximal end 14 (after removing disposable sheath 28) for placement into a patient once the clinician has located the distal end 16 of the guide wire 10 to a desired location in a patient.

The guiding insert should be maintained in a sterile condition prior to and during placements, which requires adherence to suitable procedures.

In one example, a sterile sheath 28 is assembled over the proximal end of the guiding insert, which is the end that is intended to reside in the contact-less inductive coupler assembly 20 associated with a signal receiver or generator apparatus 22. The sheath, which is placed inside the contact-less inductive coupler assembly, protects the sterility of the electromagnetic-radiation-element inside it. When the sheath is removed in keeping with maintenance of an aseptic technique, the guiding insert is not contaminated as the result of its being inserted into the non-contact inductive coupler. The material, length and shape of the sheath will be as are required for like products, because similar sheathing requirements are known for unrelated products and devices.

If a guide wire was placed into the coupler assembly during placement of the guide wire it would no longer be sterile after its first insertion.

If a guiding insert was placed into the inductive coupler assembly for the purposes of location, maintenance of an aseptic technique could not be achieved without such a sheath.

When catheters are being inserted into patients, guiding inserts must not become contaminated during procedures prior to catheters being placed “over the wire”.

An option disclosed herein is the use of one or more sheaths say approximately 20 cm long, whose internal diameter is slightly more than the outer diameter of the guide wire, placed over the proximal end of the guide wire prior to the insertion of the guide wire into the inductive coupler of the location device. Such sheaths are sterilised during assembly of the product. When the guide wire is removed from the location device, the outside of the sheath is no longer sterile whereupon it is discarded, away from the sterile field. Following this step in a placement procedure, the catheter is able to be threaded ‘over-the-wire’, which is still sterile because it has been protected by the sheath. Such sheaths are sterilized during assembly of the product.

Multiple sterile sheaths may be supplied because certain steps in such placement procedures may need to be repeated.

Of advantage to the clinician is the ability to determine the length of catheter 24 required to fit over a guide wire. Determination of lengths is readily achieved by noting one or more indicia or markers located along the length of a guiding insert (not shown in FIG. 3). These indicia (for example, numbers, coded letters or numbers) or markers (for example, bands/bars of colour and predetermined length) are indicative of the lengths of the guiding inserts between their distal ends and the indicia or markers.

The clinician who has noted the information provided by the indicia can assess, in conjunction with one or more other requirements, the optimal length of a catheter 24 necessary for placement in the patient.

Procedures can therefore be made available to the clinician to match the available catheters to at least a required minimum length.

Whenever this specification refers to protocols or procedures it is not assumed that a particular protocol exists but that clinicians are able to develop practices that will evolve over time for existing and new clinical procedures and as such it is understood that such protocols will reflect actual use of certain equipment in the clinical environment.

Advantageously, since the maximum outer dimension of the guide wire (including the proximal end electromagnetic-radiation-element 14 as well as the conductive wire arrangement 16) is sized to be smaller than the minimum inner dimension of the catheter 24, the catheter can be readily fitted over the guide wire. Thus the distal end 26 of the catheter 24 can be guided by the guiding insert to the desired location in the patient. Furthermore, since the diameter of the distal end of the guiding insert 12 is the same or smaller than the maximum outer dimension of the guiding insert as described herein, the catheter fits over it as well, allowing the distal end of the catheter to be located at the desired location in the patient and for the guiding insert to be retracted from the catheter.

FIG. 4 depicts an ‘over-the-guide wire’ placement of a catheter while the distal end of the guiding insert is at a desired location within the patient and the guiding insert is placed in the inductive coupler assembly 20. This step may not be necessary in all instances, since placement of some catheters may be verified as being in the desired location by a combination of knowledge of appropriate catheter length and the skill of a clinician in each instance. However, since is it possible to place the proximal end 14 of the guide wire 10 into the location equipment 22 (FIG. 2) via the inductive coupler assembly 20 (also FIG. 2), the guiding insert 10 can be used again to confirm location of its distal end 12 while the catheter is in situ over the guide wire. The sheath 28 previously described remains over the proximal end, as the contact-less design of the guide wire and inductive coupler assembly 20 of the location apparatus is not affected by the presence of the sheath.

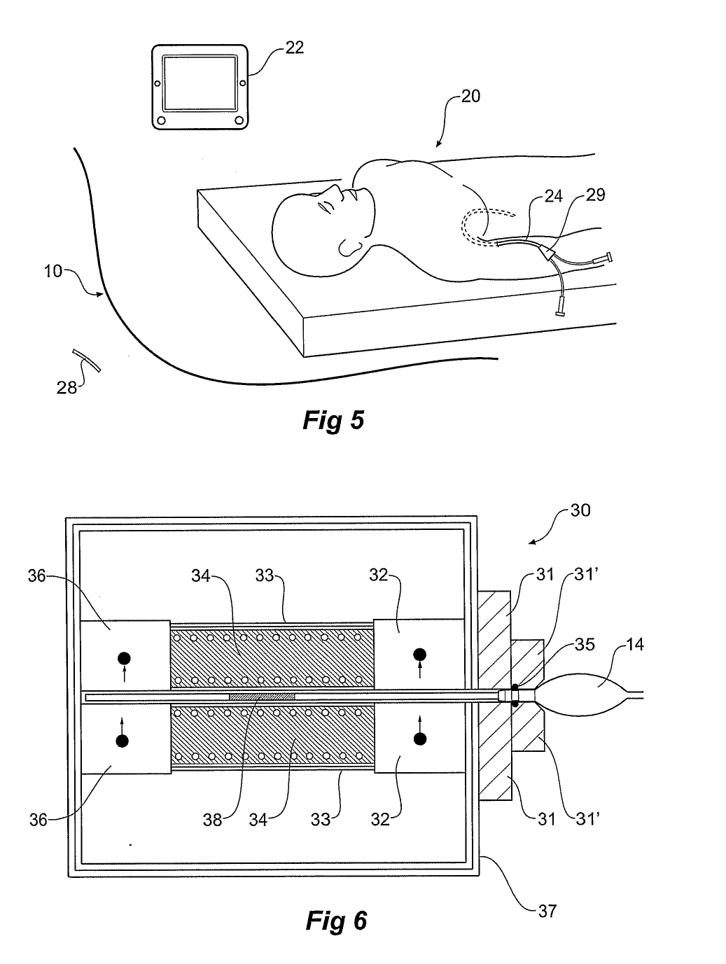

FIG. 5 depicts a catheter in place within a patient ready for use and the retracted guide wire 10. The proximal end 27 of the catheter is terminated with a Y-port 29 that allows for connection of at least two further tubes. The Y-port may also have plugs (not shown) to seal off the open ends of the two ports. Such a terminating arrangement is merely an example, and indeed many varied termination arrangements are possible.

FIG. 6 depicts a functional illustration in cross-section of one embodiment of an arrangement to locate the proximal end of a guiding insert in an inductive coupler 37 which is part of a locator device 22 (not shown), wherein the locator device is used to indicate the location of the distal end of the guiding insert in a patient.

The arrangement depicted in FIG. 6 is of an inductive coupler associated with a signal receiver or generator apparatus which accepts and locates the proximally located electromagnetic-radiation-element of the guide wire to permit inductive exchange of electromagnetic energy between the coupler and the radiation element proximally located on the guide wire.

In the functional arrangement depicted, an inlet assembly is located on the exterior wall of the locator device 22 (not shown), a central opening of the inlet assembly is sized to allow the entry of the proximal end 14 of the guide wire 10.

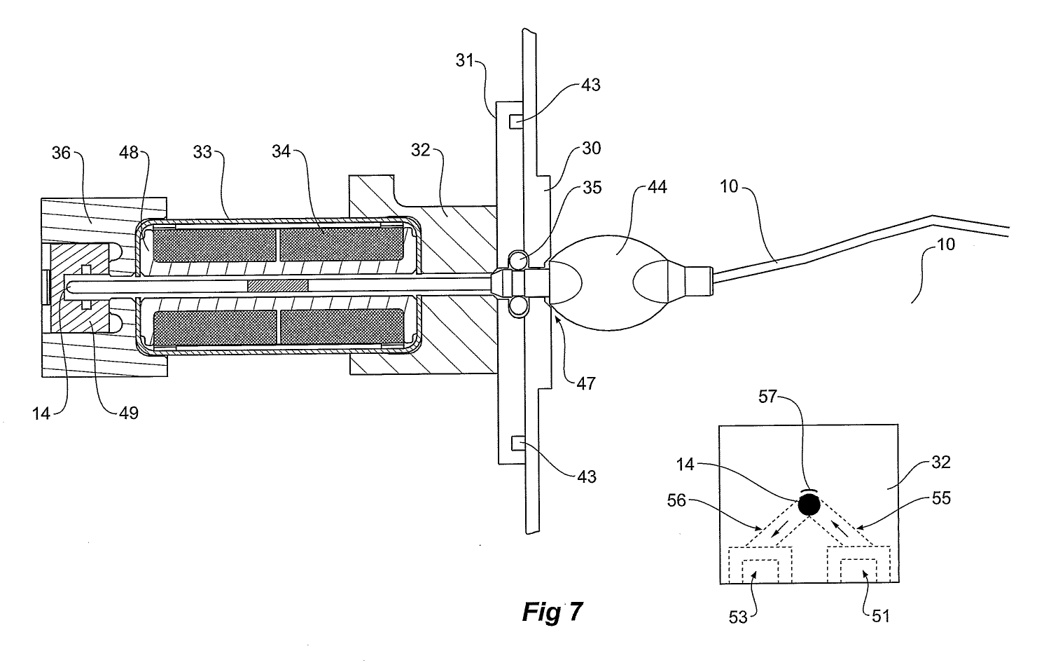

The inlet assembly is shown functionally as including a plate 31 as part of the inlet assembly 30 located adjacent and coaxial with an opening into the locator device 22 (not shown). A second plate 31′ also has a channel there through and is also adapted, by being shaped, to position an “O” ring 35 adjacent a channel and surround the proximal end of a guiding insert when it is inserted through the coaxial channels of both the plates 31 and 31′ to enter into the locator device 37. The “O” ring 35 forms a slidable interference fit with the outer wall of the proximal end of the guiding insert which is slidable through the “O” ring and once inserted held within the channel and the locator device 22. The slidable interference fit between the “O” ring 35 and the proximal end of the guide wire assembly also provides tactile feedback to the operator and that the proximal end of the guide wire is positioned correctly in the locator device. Also refer FIG. 7 for a detailed view of an “O” ring placement in particular embodiment.

While the proximal end of the guide wire 14 is being inserted into the device, measurements can be made and an indication provided to the user, which will provide confirmation that the radiation element 38 has achieved maximum mutual coupling of electromagnetic energy between the driver coil 34 and the proximally located radiation element 38.

The blocks 32 shown on the inside of the locator device 22 represent at least a proximity sensor that can detect the presence of the proximal end of the guiding insert as it passes through the inlet assembly 30 into the locator device 22, wherein such detection can then confirm that the guiding insert is entering the device 22 in the intended manner.

The blocks 32 may alternatively or additionally, detect the type of guide wire that has been placed through the inductive coupler inlet aperture. This capability can be achieved in a multitude of ways, some examples of which are the detection of indicia or markings located near the proximal end of the guiding insert. There may be one or more passive or active devices on or embedded in the guiding insert that interact with the blocks 32 in a way that identify characteristics that correlate with specifications of particular guiding inserts.

Information that identifies the guide wire type and hence its specification can assist with stock control processes, selection and traceability of materials used during surgical procedures, assist in the audit processes to ensure adherence to institutional protocols and policies such as single use of disposable devices, and most helpfully, automatic operation of the locator device in manner that simplifies the process of choosing the most appropriate type of guiding insert for each patient. There may also be, but not illustrated, a mechanism or electronic device, for example, a fusible link which if triggered appropriately indicates that the guiding insert has been used previously. Suitable protocols will then determined what is done next, but may include disposal of the guide wire as the equipment will be programmed not to work with that particular guide wire.

One characteristic that would advantageously be controllable, knowing the type of guide wire being used, is the power that needs to be used to efficiently deliver signal via the proximal electromagnetic-radiation-element to the distal electromagnetic-radiation-element, since the size of the respective elements can greatly affect their ability to receive/transmit electromagnetic energy. A coil of small dimension (being one example of an electromagnetic-radiation-element), such as may be used in a paediatric guiding insert, will require more electromagnetic energy to be delivered to it compared with the energy required to produce an equivalent strength of signal from a larger coil as might be used in an adult vascular location application.

Block 34 is the driver coil of the inductive coupler assembly 20 that is used to transfer and/or receive electromagnetic energy to and from the electromagnetic-radiation-element located at the proximal end of the guide wire 10. Since the transfer is contact-less, the inductive coupler assembly does not have to contact in any way the end of the guiding insert. The constructional and operational details of embodiments of an inductive coupler assembly 20 will be described in greater detail later in the specification.

It is important to realise that the inductive coupler is capable of being used to transfer or receive electromagnetic energy as the application requires. In relation to the use of the guiding insert location equipment disclosed in the patents referred to that are owned by the present applicant, the inductive coupler is used to transfer electromagnetic energy to the a proximally located electromagnetic-radiation-element of the guide wire, so that the distally located electromagnetic-radiation-element can radiate a signal that assists in the location determination of the location of the distal end of the guiding insert in a patient.

The blocks 36 are the end-of-travel detectors that sense, without contact, the traversal of a proximal end of a guiding insert past the driver coil 34 but still within inductive coupler assembly 20. With detection of this type, the movement of a guiding insert into and past the driver coil can be notified to the user who is performing the insertion, hence when used as intended, the appropriate positioning of the proximal end of the guide wire will result in the end being located so as to maximise electromagnetic energy transfer between the proximally located electromagnetic-radiation-element and the driver coil.

The end-of-travel detectors in one embodiment includes an optical transmission sensor which comprises a visible light transmitter and a visible light receiver and associated circuitry to sense the correct location of the proximal end of the guide wire by detecting that the visible light has been blocked by the tip of the proximal end of the of the guide wire moving across the visible light receiver.

It is important to realize that stray magnetic flux from the inductive coupler assembly should not interfere with the distally generated electromagnetic flux used to locate the distal end of the guiding insert in a patient. The magnetic circuit of the inductive coupler can therefore be shielded to prevent escape of any stray magnetic flux generated by the inductive coupler coil which is designed to minimise that occurrence in the first instance. In one particular embodiment, the driver coil 34 is encased in a magnetic shield 33 comprising two layers of high permeability foil sheet. The inductive coupler assembly case 37 forms a second outer magnetic shield and is also preferably formed from high permeability structural sheet plus high permeability foil is used in the inner surfaces wherein all joins are conductively sealed.

FIG. 7 depicts a detailed cross-sectional illustration of an embodiment of the arrangement to locate the proximal end 14 of the guide wire 10 within the inductive coupler assembly 20 of as compared with the more functional illustration of FIG. 6.

An adapter plate 30 provides an inlet to the inductive coupler assembly 20. The adapter plate 30 mates to the front panel 31 of the locator unit 20 via the locating pins 43, and in this particular embodiment the adapter plate 30 also holds the “O” ring which is partially located in a suitably shaped portion of the front panel 31 of the locator unit. The “O” ring as stated previously provides a means to impart tactile feedback to the user inserting the guide wire and also retains the proximal end of the guiding insert in the locator unit 20 once it is inserted, preventing it from accidentally falling out. A finger grip portion 44 is moulded on to the end of the guiding insert and assists the user to hold and place the proximal end of the guide wire as it is placed into the locator unit 20. The finger grip portion 44 at 47 also provides an end stop which prevents the guiding insert being pushed further into the locator unit than required.

A cross sectional detail is shown in FIG. 7A of the proximity and colour sensor block 32. A tri-colour transmitter Light Emitting Diode (LED) 51 shines light through a light conductive element 55 which is reflected from the body of the guiding insert assembly 10 via a second light conductive element 56 to a light detecting sensor 53, the reflected light is the result of the filtering effect the outer body of the guiding insert assembly 10 permitting the body (and thus guiding insert type) to be identified by the sensor which is capable of detecting more than one colour and thus in this embodiment having detected a particular colour the device can associate that colour with a particular type of guiding insert assembly. It is helpful that a guiding insert assembly is identifiable otherwise some of the automatic settings for controlling the operation of the guiding insert assembly will be inappropriate and the result could be incorrect positional output.

Two additional features of the visible light detection system include, that the solid surface at region 57 is coloured white to reflect all colours; this allows the colour sense system to positively know that the guiding insert has been inserted or removed from the inductive coupler assembly case. Further, the system can test for particular component failures by switching the transmitter LED to each primary colour in turn and verifying that each is functional by detecting an appropriate response at the sensor 53.

A further mode of operation of the sensor block 32 is to monitor changes in colour or brightness as the body of the guiding insert assembly is inserted into the inductive coupler assembly case. This allows information that is encoded on the guiding insert body by in one example, a series of coloured bands, to be read and interpreted as required.

The inductive coupler associated with a signal receiver or generator apparatus is provided in one embodiment as an inductor element, such a coil of wire wound on a bobbin 48. This is but one alternative device useable for exchanging electromagnetic energy with the device proximally located on the catheter. The bobbin 48 acts as a former about which wire can be coiled and is useable to transfer electromagnetic energy into and collect electromagnetic energy from the proximally located electromagnetic-radiation-element of the guide wire 10. More detail about the inductor bobbin and coil winding will be provided later in the specification.

All the elements 32, 33, 36, 40, 48, and 44 have a central passage for the guiding insert 10 to pass through, allowing the extreme proximal end of the guiding insert to pass into block 36 which includes the transmissive photo-micro sensor 49 described previously to provide an end-of-travel detector arrangement.

A shield 33 is located about the bobbin and around its ends having an aperture to permit entry of the proximally located end of the guide wire 10 into the electromagnetically shielded enclosure. The shield 33 which has been described previously includes in this embodiment two layers of high permeability foil electrically isolated from each other by a thin layer of insulation material.

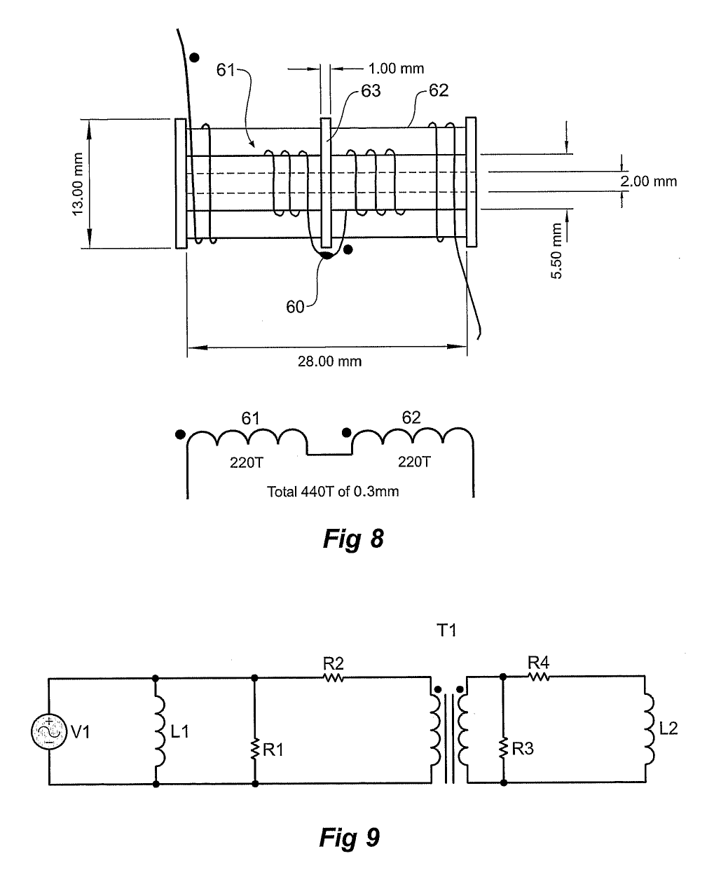

FIG. 8 depicts an embodiment of the a driver coil, wherein the driver coil is a single ended coil which is formed by two halves 61 & 62 which have common point 60 at two the inner ends of the two halves and which is driven at the outer ends of the two halves of the coils. The two coil winding halves are wound in opposite directions and a central barrier 63 is used to allow each half of the winding to be wound separately.

The configuration of the coils ensures there is a distance across the coil diameter between the outer coil layers immediately connected to the drive nodes, which minimizes capacitive coupling of common mode voltage between the drive nodes and the electromagnetic-radiation-element of the guide wire. The configuration also ensures that the common mode drive voltage appearing on the innermost winding layer which is closest to the electromagnetic-radiation-element is at a minimum. The coil design described reduces the amplitude of common mode synchronous signals on any conductive parts of the guiding insert assembly, otherwise these signals would radiate into the signal detection system and possibly cause errors in the position measurement, particularly at long distances where magnetic signal strength is low and the unwanted radiated signal may to dominate.

The physical dimensions indicated, inductance values, wire turns and wire gauge are as follows in this embodiment and when provided the required signal strength output, coupling factor, efficiency and accuracy will be available to drive the position detection function of the system and the assembly.

FIG. 9 depicts an equivalent circuit to an inductive coupler arrangement wherein the electromagnetic energy transfer acts like a lossy transformer and coupling calculations treat the relevant elements accordingly.

The use of the terms “driver” coil and “coupling” coil in the figures are relevant to but one version of the possible purposes of the coils, as their functions can be reversed in alternate embodiments of the invention.

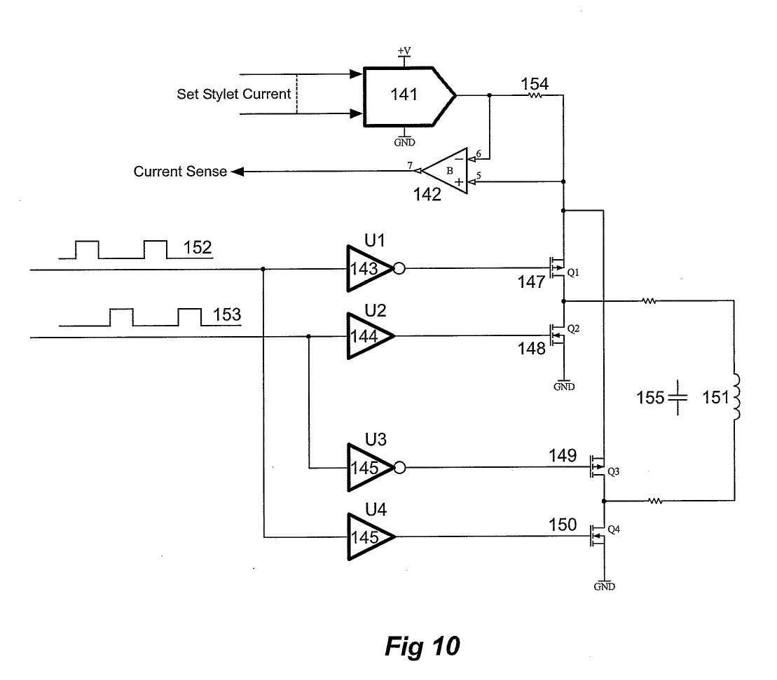

FIG. 10 depicts a signal driver circuit for supplying a signal to the inductive coupler that provides a signal for electromagnetic radiation from the distal end of the guide wire.

The drive to the coil must be symmetrical in impedance and timing otherwise errors may result at positions where the electromagnetic signal phase reverses due to changing angular orientation of the distally located electromagnetic-radiation-element of the guide wire in relation to the receiver coil. For the drive to the coil to be symmetrical in impedance and timing a dual half bridge driver comprising elements 143 to 150 as depicted in the circuit of FIG. 10 are used. The signal amplitude output to the drive coil 151 is controlled by the variable DC voltage source 141. The signal inputs 152 & 153 comprise two non-overlapping time symmetrical drive signals which synchronise output from the two half bridge drives. The supply current to the half bridge drivers is monitored by a current sensor comprising the current sense resistor 154 and amplifier 142.

The current sensor circuit provides an output proportional to the driver coil current. By monitoring the driver coil current and driver coil voltage, and performing suitable computations, a number of aims can be achieved:
a) The amount of energy driven into the secondary and hence into the transmitter coil can be controlled. This allows the driver to accommodate different guiding inserts for different purposes (such as a smaller guide wire for paediatric use);
b) The driver can compensate to some extent for variations in the resistance of the guiding inserts caused by fluctuations in the manufacturing process, and
c) The impedance of the secondary circuit can be inferred, with this allowing for the detection of characteristics of the guiding inserts such as open circuit and short circuit conditions.

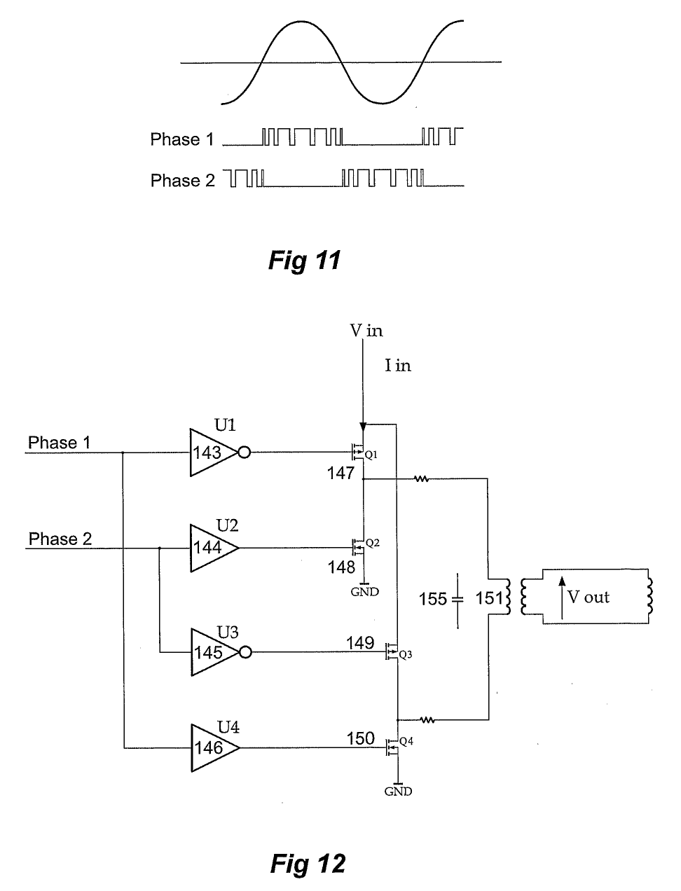

FIG. 11 depicts a pulse width modulation timing scheme suitable for driving the electromagnetic-radiation-element driver coil 151 using the circuit of FIG. 12 that in the example given aims to produce a substantially sinusoidal waveform at a required frequency.

It is preferable to drive the primary coil with a sinusoidal excitation as this will have a number of benefits:
a) Minimising EMI by reducing or eliminating unwanted harmonics;
b) Maximising efficiency for the same reason as above;
c) Reducing eddy current heating in the secondary and transmitter coils, again for the same reason;

One way of achieving this is to use a parallel capacitor 155 to form a resonant circuit with the inductance of the driver coil 151 as also depicted in FIG. 10.

There are however disadvantages in such an arrangement:
a) it is relevant to a fixed frequency of operation, and circuit valued must be adjusted if frequency is to change;
b) component values depend on frequency of operation and physical shape/disposition of the primary coil and this may require component values that are not readily or even available;
c) the signal driver can not automatically adapt to variations in the primary coil caused by variations in manufacturing of the components;
d) and specific to the embodiments disclosed herein, the Q of the tuned circuit can not be made too high, otherwise there may be adverse effects on the gain and phase stability.

Thus use of a PWM scheme, as disclosed in association with FIG. 12 can assist in reducing or eliminating the issues described by enabling operation at frequencies insensitive to manufacturing variation.

FIG. 12 depicts a pulse width modulation signal driving circuit for use with an embodiment of the invention which works in a similar manner to that described in relation to the circuit depicted in FIG. 10.

It will be appreciated by those skilled in the art that the invention is not restricted in its use to the particular application described. Neither is the present invention restricted in its preferred embodiment with regard to the particular elements and/or features described or depicted herein. It will be appreciated that various modifications can be made without departing from the principles of the invention. Therefore, the invention should be understood to include all such modifications within its scope.

1. A guiding insert assembly having a distal end and a proximal end including
a distally located electromagnetic-radiation-element;
a proximally located electromagnetic-radiation-element; and
a conductive wire arrangement connecting the distally located electromagnetic-radiation-element to the proximally located electromagnetic-radiation-element.

2. A guiding insert signal inductive coupling arrangement including
a guiding insert assembly having a distal end and a proximal end including
a distally located electromagnetic-radiation-element;
a proximally located electromagnetic-radiation-element; and
a conductive wire arrangement connecting the distally located electromagnetic-radiation-element to the proximally located electromagnetic-radiation-element; and
an inductive coupler associated with a signal receiver or generator apparatus adapted to receive the proximal end of the guiding wire to permit inductive exchange of electromagnetic energy between the coupler and the proximally located radiation element for transmission to the distally located electromagnetic-radiation-element.

3. The guiding insert assembly according to claim 1 wherein the maximum outer diameter of both electromagnetic-radiation-elements is substantially the same.

4. The guiding insert assembly according to claim 1 wherein the maximum outer diameter of the conductive wire arrangement is less than the maximum outer diameter of both electromagnetic-radiation-elements.

5. The guiding insert assembly according to claim 1 wherein the maximum outer diameter of either of the electromagnetic-radiation-elements is sized so as to allow a catheter to pass over at least the proximally located electromagnetic-radiation-element or both of the electromagnetic-radiation-elements.

6. The guiding insert assembly according to claim 1 wherein the distal end of the guiding wire assembly is atraumatic in its effect upon the tissue of a patient in whom the guiding wire assembly is placed.

7. The guiding insert assembly according to claim 1 further includes at either or both the proximal end and distal end an electromagnetically-powered identification device.

8. The guiding insert assembly according to claim 1 further includes indicia along at least a portion of the length of the conductive wire arrangement to indicate the distance from the distal end as a gauge of the location of the distally located electromagnetic-radiation-element.

9. The guiding insert assembly according to claim 8 further includes indicia on the proximal end that identifies a type of guiding insert assembly.

10. The guiding insert assembly according to claim 1 further includes a position detector for the proximal end of the guiding insert assembly.

11. The guiding insert signal inductive coupling arrangement of claim 2 further including a position detector on the proximal end of the guiding insert assembly.

12. The guiding insert assembly according to claim 10 wherein the position detector provides a human detectable signal of the positioning of the proximally located electromagnetic-radiation-element so as to permit inductive exchange of electromagnetic energy between the coupler and the proximally located electromagnetic-radiation-element.

13. The guiding insert signal inductive coupling arrangement according to claim 2 wherein the inductive coupler further includes a measuring device that detects a voltage or current of the inductive coupler device to provide an indication of predetermined characteristics of the guiding insert assembly.

14. The guiding insert signal inductive coupling arrangement according to claim 13 wherein the predetermined characteristics include:
guiding wire material type;
resistance of conductive wire assembly; or
location of the proximally located electromagnetic-radiation-element with respect to the inductive coupler device.

15. The guiding insert signal inductive coupling arrangement according to claim 2 wherein the inductive coupler further includes:
an impedance measuring device that detects an abnormality in the guiding wire assembly, including an open circuit or short circuit of the inductive coupler or with the electromagnetic-radiation-element in the guiding insert assembly.

16. The guiding insert signal inductive coupling arrangement according to claim 2 wherein the inductive coupler further includes:
an impedance measuring device that detects a characteristic of the electromagnetic-radiation-element of the guiding wire assembly, indicative of the efficacy of coupling of the inductive coupler with the inductive coupling.

17. The guiding insert signal inductive coupling arrangement according to claim 2 wherein the inductive coupler further includes:
an impedance measuring device that detects an impedance related abnormality in the material of the guiding insert assembly.

18. The guiding insert signal inductive coupling arrangement according to claim 15 wherein if an abnormality is detected by the impedance measuring device, the inductive coupler ceases inductive exchange of electromagnetic energy between the coupler and the radiation element.

19. The guiding insert signal inductive coupling arrangement according to claim 16 wherein if an abnormality is detected by the impedance measuring device, the inductive coupler ceases inductive exchange of electromagnetic energy between the coupler and the radiation element.

20. The guiding insert signal inductive coupling arrangement according to claim 17 wherein if an abnormality is detected by the impedance measuring device, the inductive coupler ceases inductive exchange of electromagnetic energy between the coupler and the radiation element.
